(19) United States
(12) Patent Application Publication
(10) Pub. No.: US 2023/0172972 A1
(43) Pub. Date: Jun. 8, 2023

(54) OPHTHALMIC COMPOSITION AND METHOD OF USE

(71) Applicant: Carl C. Awh, Nashville, TN (US)

(72) Inventor: Carl C. Awh, Nashville, TN (US)

(21) Appl. No.: 17/911,958

(22) PCT Filed: Dec. 4, 2020
(86) PCT No.: PCT/US2020/063355

§ 371 Date: Sep. 15, 2022

Related U.S. Application Data
(60) Provisional application No. 62/990,178, filed on Mar. 16, 2020.

(51) Int. Cl. A61K 31/79; A61K 31/245; A61K 9/00; A61K 9/06

(52) U.S. Cl. CPC A61K 31/79; A61K 31/245; A61K 9/0048; A61K 9/06

(57) ABSTRACT

Ophthalmic compositions for administration to an eye including a mixture of an antiseptic and an anesthetic in therapeutically effective amounts are disclosed. The compositions are both anesthetic and antiseptic, and can be used in various ophthalmic procedures.

### CROSS-REFERENCE TO RELATED APPLICATIONS

This application claims priority from Provisional Application No. 62/990,178, filed Mar. 16, 2020, the contents of which are hereby incorporated by reference in their entirety.

### FIELD OF THE INVENTION

The subject matter herein relates generally to ophthalmic antiseptic and anesthetic compositions for use during ophthalmic procedures.

### BACKGROUND OF THE INVENTION

Intraocular injection with the administration of various agents has become a common treatment of many ophthalmic conditions, such as age-related macular degeneration, diabetic retinopathy, endophthalmitis, viral retinitis, cystoid macular edema, uveitis, retinal vascular occlusion, and retinal detachment. For example, an intraocular injection into the vitreous cavity, commonly referred to as an intravitreal injection, is typically used in the treatment of neovascular age-related macular degeneration. Prior to the injection of a substance into the eye, the patient is typically administered multiple applications of an anesthetic (topical and sometimes subconjunctival) to provide sufficient anesthesia for patient comfort during the injection procedure. A topical antiseptic agent is also typically administered to minimize the risk of infection associated with an intraocular injection. It is reported that over 4 million intraocular injections were performed in the USA in 2013, rising further to an estimated 5.9 million injections in 2016, highlighting the need for practical compositions and methods to simplify procedures and to reduce possible risks and complications ranging from discomfort to severe complications, e.g. endophthalmitis.

The current standard of anesthesia and antisepsis prior to intraocular injection typically includes 1) the administration of proparacaine or tetracaine drops; often combined with lidocaine gel, lidocaine-soaked pledgets, or a subconjunctival lidocaine injection for anesthesia; followed by 2) the administration of polyvinylpyrrolidone (povidone) iodine (PVI) 5% drops for antisepsis followed by 3) saline irrigation to minimize the post-injection discomfort caused by the irritant properties of PVI 5% that can last more than 24 hours despite adequate topical anesthesia. However, the anesthesia and antisepsis steps are time consuming, can introduce a risk of infection after injection, and post-injection pain is often reported as a result of the irritant properties of PVI when used at a concentration of 5%. It is accepted practice that the anesthetic and PVI should be administered sequentially to allow a sufficient amount of time for the anesthetic to work, to prevent dilution of the PVI by the previously administered anesthetic, and to reduce irritation caused by the PVI when administered after the anesthetic. In fact, the accepted practice, e.g., by the Glaucoma Research Foundation, includes a waiting period of about 5 minutes between the administration of the anesthetic and administration of the PVI. Another waiting period of about 30 seconds or longer is also included to allow for a sufficient amount of contact time of the PVI for proper antisepsis of the eye. The additional step of saline irrigation further contributes to a significant amount of time to the procedure and adds a considerable amount of discomfort to the patient. In addition, both the separate administration of the anesthetic and PVI and the saline irrigation can introduce a risk of infection. Saline irrigation can liberate microbes from the conjunctival fornices, the eyelids, and other periorbital tissues. Additionally, when the anesthetic is administered in a gel formulation prior to administration of the antiseptic, an increased survival of microbes protected or sequestered by the gel formulation increases the risk of infection after injection.

Accordingly, there is a long felt but unmet need for a topical composition to simultaneously provide both effective anesthesia and antisepsis during an ophthalmic procedure with minimal discomfort to the patient and to reduce time and effort during an ophthalmic procedure.

### SUMMARY OF THE INVENTION

One aspect of the present disclosure encompasses a composition for administration to an eye. The composition comprises povidone-iodine in a concentration between about 0.01% and about 5.0% by weight; and proparacaine hydrochloride in a concentration between about 0.2% and about 0.6% by weight. The povidone-iodine can be in a concentration of about 0.9% to about 1.1% by weight, and the proparacaine hydrochloride can be in a concentration of about 0.4% to about 0.5% by weight.

The composition can further comprise an antimicrobial preservative, a co-solvent/surfactant, a viscosity increasing agent, a pharmaceutically acceptable excipient, or combinations thereof. The composition can be in the form of a solution, suspension, emulsion, ointment, cream, gel, or a controlled-release vehicle.

Another aspect of the present disclosure encompasses an ophthalmic antiseptic and anesthetic dosage form. The dosage form comprises povidone-iodine at a concentration between about 0.01% and about 5.0% by weight; and proparacaine hydrochloride at a concentration between about 0.2% and about 0.6% by weight. The dosage form can be eye drops or a gel. The povidone-iodine can be in a concentration of about 0.9% to about 1.1% by weight; and the proparacaine hydrochloride can be in a concentration of about 0.4% to about 0.5% by weight.

Yet another aspect of the present disclosure encompasses a method of treatment for an eye. The method comprises administering a therapeutically effective amount of an ophthalmic antiseptic and anesthetic composition to the eye of a subject in need thereof, wherein the composition comprises povidone-iodine in a concentration between about 0.01% and about 5.0% by weight; and proparacaine hydrochloride in a concentration between about 0.2% and about 0.6% by weight. The povidone-iodine can be in a concentration of about 0.9% to about 1.1% by weight; and the proparacaine hydrochloride can be in a concentration of about 0.4% to about 0.5% by weight. The subject can be a human subject.

An additional aspect of the present disclosure encompasses a method of relieving discomfort associated with an ophthalmic procedure. The method comprises administering a therapeutically effective amount of an ophthalmic antiseptic and anesthetic composition to the eye of a subject in need thereof, the composition comprising povidone-iodine in a concentration between about 0.01% and about 5.0% by weight; and proparacaine hydrochloride in a concentration between about 0.2% and about 0.6% by weight.

Another aspect of the present disclosure encompasses a method of anesthetizing and disinfecting an eye during an ophthalmic procedure. The method comprises administering a therapeutically effective amount of an ophthalmic antiseptic and anesthetic composition to the eye of a subject in need thereof, the composition comprising povidone-iodine in a concentration between about 0.01% and about 5.0% by weight; and proparacaine hydrochloride in a concentration between about 0.2% and about 0.6% by weight.

Yet another aspect of the present disclosure encompasses a kit comprising one or more containers comprising ophthalmic antiseptic and anesthetic composition. The composition comprises povidone-iodine in a concentration between about 0.01% and about 5.0% by weight; and proparacaine hydrochloride in a concentration between about 0.2% and about 0.6% by weight. The container can be opaque or dark-colored. The antiseptic and anesthetic can be combined in a single container. Alternatively, the antiseptic and anesthetic can be packaged in separate chambers of a container. The antiseptic and anesthetic can also be packaged in separate containers. The kit can further comprise means for administering the composition.

### DETAILED DESCRIPTION OF THE INVENTION

The present disclosure encompasses an ophthalmic antiseptic and anesthetic composition and methods of using the composition during an ophthalmic procedure. The composition comprises proparacaine anesthetic and PVI at a concentration considerably lower than the concentration of PVI used in the generally accepted treatment protocol. More specifically, the composition comprises a concentration of PVI up to 20-fold lower than the concentration of PVI used in the generally accepted treatment protocol. Surprisingly, the inventor discovered that the lower concentration of PVI provided equally or more effective antisepsis without irritating the eye even in a composition combining the anesthetic and antiseptic. Even more surprisingly, the inventor discovered that the composition is effective at simultaneously providing anesthesia and disinfection, all while significantly reducing discomfort to the subject before, during, and after the procedure when compared to methods of anesthesia and antisepsis routinely used by ophthalmologists. Advantageously, the composition reduces the time and effort required during an ophthalmic procedure by reducing the number of separate applications necessary to achieve effective anesthesia and antisepsis and by eliminating the need for saline irrigation to reduce irritation to the eye. The composition further reduces the time and effort required during an ophthalmic procedure by combining the separate waiting period required for effective anesthesia and the waiting period required for effective antisepsis into a single waiting period capable of achieving both effective anesthesia and effective antisepsis. These simplified procedures and improvement in comfort and safety of the procedures were a welcome surprise both to subjects undergoing the ophthalmic procedure and to technicians performing or assisting during the procedure.

### I. Composition

One aspect of the present disclosure encompasses an ophthalmic antiseptic and anesthetic composition for administration to an eye during an ophthalmic procedure. The composition comprises an antiseptic and an anesthetic.

In some aspects, the antiseptic is povidone-iodine (PVI). PVI has the following chemical structure:

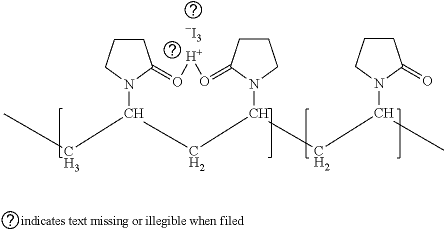

The concentration of povidone-iodine in the ophthalmic composition can range from about 0.01% to about 5.0% by weight, from about 0.5% to about 5.0% by weight, from about 0.5% to about 2.0% by weight, from about 0.01% to about 0.03%, or from about 0.7% to about 1.2% by weight. In some aspects, the concentration of povidone-iodine in the ophthalmic composition ranges from about 0.7% to about 1.2% by weight. In an alternative of the aspects, the concentration of povidone iodine in the ophthalmic composition is about 0.9% to about 1.1% by weight.

In some aspects, the anesthetic is proparacaine hydrochloride. Proparacaine hydrochloride has the following chemical structure:

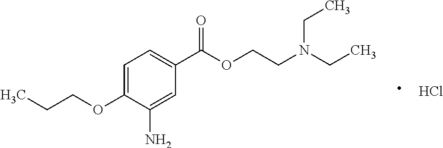 [SMILES: CCCOc1ccc(C(=O)OCCN(CC)CC)cc1N ; Cl]

The concentration of proparacaine hydrochloride in the ophthalmic composition can range from about 0.01% to about 5% by weight, from about 0.1% to about 1.0% by weight, from about 0.2% to about 0.6% by weight, or from about 0.3% to about 0.7% by weight. In some aspects, the concentration of proparacaine hydrochloride in the ophthalmic composition ranges from about 0.3% to about 0.7% by weight. In alternative aspects, the concentration of proparacaine hydrochloride in the ophthalmic composition is about 0.4% to about 0.5% by weight.

In some aspects, the composition comprises povidone-iodine in a concentration between about 0.05% and about 5.0% by weight, and proparacaine hydrochloride in a concentration between about 0.2% and about 0.6% by weight. In an aspect, the composition comprises povidone-iodine in a concentration of about 0.05% and about 1.0% by weight, and proparacaine hydrochloride in a concentration of about 0.2% and about 0.6% by weight. In an aspect, the composition comprises povidone-iodine in a concentration of about 0.9% to about 1.1% by weight, and proparacaine hydrochloride in a concentration of about 0.4% to about 0.5% by weight. In alternate aspects, the concentration of ingredients in the composition may be designated in other suitable ratio measurements, such as percentage by volume.

In alternative aspects, the antiseptic may include or combine other suitable topical anesthetics for the composition including, but not limited to proparacaine, lidocaine, tetracaine, or a derivative or combination thereof.

In alternative aspects, the anesthetic may include or combine other suitable antiseptics including, but not limited to aqueous chlorhexidine gluconate, or a derivative or combination thereof.

In any of the disclosed compositions for topical administration, such as compositions composed for topical administration to the eye, the mixtures can be formulated as a suitable percent-by-weight solution in water at a pH of 5.0 to 8.0 (figures relate to the combined presence of povidone-iodine and dexamethasone). This pH range may be achieved by the addition of buffers to the solution.

In some aspects, compositions of the present disclosure are formulated for topical administration as solutions, suspensions, emulsions (dispersions), gels, creams, or ointments in a suitable ophthalmic vehicle. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In some aspects, the compositions are formulated as a solution. In other aspects, the compositions are formulated as a gel. Unlike the anesthetic gel formulations currently used in the field, the instant gel formulations, comprising both the antiseptic and anesthetic, do not present a risk of infection after injection. In fact, gel compositions of the instant disclosure are equally effective as liquid solutions.

Various aspects of the compositions can be formulated by including one or more excipients in a formulation. Non-limiting examples of excipients include preservatives (antioxidants), tonicity agents such as NaCl, suspending agents, and coloring agents, pH modifiers, chelating agents, antimicrobial preservatives, and combinations of any of these agents.

Non-limiting examples of preservatives include ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apocarotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanillic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof.

Compositions optionally include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and natural and derived colorants may be used in compositions encompassed by the present disclosure.

Non-limiting examples of pH modifiers include citric acid, acetic acid, tartaric acid, malic acid, fumaric acid, lactic acid, phosphoric acid, sorbic acid, benzoic acid, potassium and sodium phosphate, sodium carbonate and sodium bicarbonate.

A chelating agent may be included in any of the compositions as a stabilizing excipient to complex heavy metals that might promote instability. Non-limiting examples of chelating agents include lysine, methionine, glycine, gluconate, polysaccharides, glutamate, aspartate, and disodium ethylenediaminetetraacetate (Na₂EDTA).

An antimicrobial agent may be included in any of the compositions as an excipient to minimize the degradation of the compound according to this disclosure by microbial agents, including but not limited to bacteria and fungi. Non-limiting examples of antimicrobials include benzalkonium chloride, thimerosal, chlorobutanol, para-bens such as methylparaben, propylparaben, phenylethyl alcohol, EDTA, sorbic acid, Onamer M, phenol, calcium propionate, sodium nitrate, sodium nitrite, and sulfites including, but not limited to sulfur dioxide, sodium bisulfite, and potassium hydrogen sulfite. Generally, such antimicrobial agents are employed at a concentration level of from 0.001% to 1.0% by weight.

A composition may optionally contain a co-solvent or surfactant to enhance the solubility of the components of the composition. For example, the co-solvent or surfactant may include polysorbate 20, 60, and 80, polyoxyethylene/polyoxypropylene surfactants (e.g. Pluronic F-68, F-84, and P-103), cyclodextrin, tyloxapol, and the like, or a combination thereof. Generally, such preservatives are employed at a concentration level of from 0.01% to 2.0% by weight.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. When the composition is formulated as a gel, the composition can further comprise a gelling agent. Gelling agents are material used to thicken & stabilize liquid solutions. They dissolve in the liquid as a colloid mixture that forms an internal structure giving the resulting gel an appearance of a solid matter. Non-limiting examples of gelling agents include Acacia, Pectin, Starch, Tragacanth, Xanthan gum, Alginic acid (seaweed), Animal/vegetable fats such as lard, cocoa butter, Gelatin, Bentonite, veegum (magnesium aluminum silicate), Carboxymethylcellulose (CMC) and other cellulose derivatives such as methyl cellulose, hydroxypropyl methylcellulose, hydroxyethyl cellulose, carboxymethyl cellulose, hydroxypropyl cellulose, Carbomer resins (carbopols), Colloidal silicon dioxide, Polyvinyl alcohol (PVA), Petrolatum, mineral oil/polyethylene gel, and combinations thereof.

### II. Methods

One aspect of the present disclosure encompasses a method of treatment for an eye. The method comprises administering a therapeutically effective amount of an ophthalmic composition to the eye of a subject in need thereof. Another aspect of the present disclosure encompasses a method of relieving discomfort associated with an ophthalmic procedure. The method comprises administering a therapeutically effective amount of an ophthalmic composition to the eye of a subject in need thereof. An additional aspect of the present disclosure encompasses a method of anesthetizing and disinfecting an eye during an ophthalmic procedure. The method comprises administering a therapeutically effective amount of an ophthalmic composition to the eye of a subject in need thereof. In all aspects of the disclosure, the composition can be as described in Section I above.

The subject can be a human, a livestock animal, a companion animal, a lab animal, or a zoological animal. In one aspect, the subject may be a rodent, e.g. a mouse, a rat, a guinea pig, etc. Non-limiting examples of suitable livestock animals may include pigs, cows, horses, goats, sheep, llamas, and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc. In some aspects the subject is a human subject.

In some aspects, the composition is in liquid form and can be administered using any spray or aerosol bottle, or by instillation (generally in liquid form drop by drop) using a plastic applicator, syringe, dropper, pipette, tube, or the like. For instance, the composition can be pre-packaged in single-use tubes. Alternatively, the composition can be administered as an eye drop using any of the many types of eye drop dispensers on the market. In an alternate aspect, the composition may be administered by applying the solution to an applicator, such as a sponge, and placing the applicator in contact with the eye. The choice of a method of administration can and will vary depending on the physical characteristics of the composition, such as viscosity, the ophthalmic procedure during which the composition is administered, and the preference of the technician and/or subject among other variables.

In an aspect, the compositions of the present disclosure are administered topically. When the composition is in an eye drop dosage form, dosage for one eye is understood to be about one drop of solution. One drop of the solution may be between 10 μl to 200 μl, between 20 μl and 120 μl, or between about 50 μl (microliters) to about 80 μl of solution, or any values in between. For example, dispensers such as pipettors can dispense fluid drops from at least 1 μl to 300 μl and any value in between.

The packaging presentation of medicines is a critical step in maintaining chemical and physical stability. For instance, active ingredients can be packaged in opaque or dark-colored containers such as amber colored containers to protect light-sensitive drug ingredients. Accordingly, the choice of packaging for a composition of the instant disclosure will be apparent to an individual of skill in the art. The container for the composition of the disclosure may be clear, translucent, dark-colored, or opaque and may contain other properties or combinations of properties such as being glass-lined, tamper-proof, packaged in single or multiple dose aliquots, and a combination thereof. In some aspects, the packing for a composition of the instant disclosure is opaque or dark-colored containers.

Proparacaine can be light-sensitive. Accordingly, in some aspects, when the anesthetic is proparacaine, the composition of the instant disclosure is packaged in opaque or dark-colored containers.

In some aspects, the composition is combined in a package for immediate use. In an alternate aspect, the container may store one or more components of the composition in separate chambers, which are combined prior to the use of the combined composition. In an alternate aspect, the components of the composition are in separate containers, and are combined prior to the use of the combined composition.

### III. Kits

A further aspect of the present disclosure provides kits comprising the antiseptic compositions detailed above in Section I. The compositions can be packaged in one or more container. The one or more containers can be opaque or dark-colored containers to protect light-sensitive drug ingredients. In one aspect, when the anesthetic is proparacaine, the container is opaque or dark-colored to protect the proparacaine from exposure to light. In some aspects, the compositions in the kit are combined in a single container for immediate use. In alternate aspects, the anesthetic and the antiseptic are packaged in separate chambers of a container, which are combined prior to the use of the combined composition. In an alternate aspect, the anesthetic and the antiseptic are packaged in separate containers, and are combined prior to the use of the combined composition.

The kits may further comprise means for administering the compositions. For instance, the kit can further comprise a dropper for administering a liquid formulation of the composition, a pledget to be soaked in the liquid composition, or a tube for administering a gel formulation of the compositions.

The kits provided herein generally include instructions for carrying out the methods detailed below. The instructions can comprise storage and expiration date information. For instance, when the anesthetic is proparacaine, the instructions include instructions for storage of the compositions at low temperatures and an expiration date to provide maximum efficacy of the anesthetic. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips) and optical media (e.g., CD ROM), and the like. As used herein, the term “instructions” may include a QR code or the address of an internet site that provides the instructions.

It is to be understood that the above description is intended to be illustrative, and not restrictive. For example, the above-described aspects (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain aspects, and are by no means limiting and are merely exemplary aspects. Many other aspects and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “ophthalmic procedure” refers to any procedure performed to the eye, during which an antiseptic and anesthetic is used. Non-limiting examples of ophthalmic procedures are refractive types of ophthalmic surgery such as Intacs, ICRs, or Intracorneal Ring, Conductive Keratoplasty (CK), Laser Assisted In Situ Keratomileusis (LASIK), Laser Assisted Subepithelial Keratomileusis (LASEK) or Epi-LASIK, and Photorefractive Keratectomy (PRK); cataract types of eye procedures such as Extracapsular Cataract Extraction (ECCE), Intracapsular Cataract Extraction (ICCE), Laser-Assisted Cataract Surgery (LACS), Phacoemulsification or Phaco, and Cryoanalgesia; corneal types of eye surgery such as Pterygium Excision, Phototherapeutic Keratectomy (PTK), and Penetrating keratoplasty (PK); oculoplastic types of ophthalmic procedures such as blepharoplasty or browplasty; and glaucoma types of surgeries. Other examples of ophthalmic procedure include anterior chamber paracentesis (a procedure to remove fluid from the anterior chamber of the eye), removal of corneal foreign bodies, contact tonometry, and removal of conjunctival sutures.

The term “anesthetic” as used herein refers to a drug used to induce anesthesia—in other words, to result in a temporary loss of sensation or awareness.

The term “antiseptic” as used herein refers to antimicrobial substances that are applied to living tissue/skin to destroy microorganisms, to prevent or inhibit the growth of microorganisms, and to reduce the possibility of infection, sepsis, or putrefaction. Advantageously, PVI has been shown to also destroy viruses, including the SARS-CoV-2 virus that causes COVID-19. This virus has been shown to infect the eyes causing conjunctivitis in a significant number of patients.

As used herein, the term “therapeutically effective amount” of an active pharmaceutical ingredient refers to an amount of the ingredient sufficient to produce a measurable desired effect. Actual dosage levels of active ingredients in a therapeutic composition of the invention may be varied so as to administer an amount of the active ingredient(s) that is effective to achieve the desired therapeutic response for a particular subject.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “treating,” “treatment,” or “to treat” each may mean to alleviate, suppress, repress, eliminate, prevent or slow the appearance of symptoms, clinical signs, or underlying pathology of a condition or disorder on a temporary or permanent basis. Preventing a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment may reduce the risk of developing the condition and/or lessen its severity if the condition later develops. For instance, treatment of a microbial infection may reduce, ameliorate, or altogether eliminate the infection, or prevent it from worsening.

The term “about” denotes a range of value ranging from −10% of the value it modifies to +10% of the value it modifies, with the proviso that such claim construction does not preclude application of broader equivalent values.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Efficacy of an Ophthalmic Composition Comprising Proparacaine 0.5% and Povidone Iodine 1.0% as a Topical Antiseptic

The efficacy of an ophthalmic solution (“Povicaine®”) containing approximately 0.5% proparacaine and 1.0% povidone iodine compared to conventional povidone iodine 5.0% (Betadine) as a topical antiseptic prior to an intraocular injection was evaluated. Standard technique using the BD ESwab Collection and Transport System was used to culture the conjunctival surface prior to administration of any topical drops. Eyes were randomly assigned to receive either Betadine or Povicaine®, with 38 eyes assigned to each solution (76 eyes in total). A second culture of the conjunctival surface was performed following standardized administration of either topical Betadine or Povicaine®. Standard linear inoculation of blood and chocolate agar was used to determine the presence or absence of bacterial growth by 72 hours.

36 eyes demonstrated bacterial growth prior to the administration of any drops (Table 1). Of these, half (n=18) had received treatment with Betadine; and half, (n=18) had received treatment with Povicaine®. Of the 18 eyes treated with Betadine, 13 (72%) exhibited no post-treatment bacterial growth. Of the 18 patients treated with Povicaine®, 15 (83%) exhibited no post-treatment bacterial growth. The results of a Fisher's Exact test* showed no significant difference in post-treatment bacterial growth between patients treated with Betadine and Povicaine® (p=0.69), with a trend toward superior outcomes with Povicaine®. Analysis was performed using SPSS Version 27.

TABLE 1
Post-treatment results

| | Betadine | Povicaine ® | |
|---|---|---|---|
| Treatment | (n = 18) | (n = 18) | P-value |
| No post-treatment growth | 13 (72%) | 15 (83%) | 0.69 |
| Post-treatment growth | 5 (28%) | 3 (17%) | |

### Example 2. Efficacy of Povicaine™ as a Topical Antiseptic

Povicaine® was applied in the form of eye drops to the eye of subjects for anesthesia and antisepsis prior to undergoing intravitreal injections. In all, about 2400 injection procedures were performed. There were no (0) cases of endophthalmitis (infection) noted or reported for any patient in this series of injections.

### Example 3. Patient Preference for Methods of Anesthetizing and Disinfecting Eyes During Ophthalmic Procedures

Povicaine® was applied in the form of eye drops to the eye of subjects for anesthesia and antisepsis prior to undergoing intravitreal injections. All patients had previously undergone ophthalmic procedures using multiple standard methods of anesthesia and antisepsis. The standard methods include the use of anesthesia with lidocaine gel, subconjunctival injection of lidocaine solution, or lidocaine-soaked pledgets; plus antisepsis with povidone iodine 5%; followed by saline irrigation (to minimize post-injection discomfort due to the povidone iodine 5%). The reactions of the patients were surveyed in regards to comfort and preference for Povicaine® when compared to the standard methods of anesthesia and antisepsis they had previously received. The survey is shown below.

In short, the patients were pleasantly surprised that Povicaine® was at least as effective at preventing pain as the anesthetic compositions used during treatments they had previously received, all while almost completely eliminating the discomfort and irritation to the eye they experienced before, during, and after the prior treatments. Patients surprisingly reported that Povicaine® is more comfortable prior to the intraocular injection, offers equivalent anesthesia during the injection, and is more comfortable following the injection. Use of the composition eliminates the need to irrigate the eye with saline solution, saving the discomfort of the irrigation procedure, with saline solution running over the patient's eyelids, and eliminates the blurred vision often associated with topical anesthetic gel currently in use in the field. More specifically, an overwhelming number of the patients reported no significant difference in the discomfort of the eye injection, whether Povicaine® or the standard method were used, demonstrating the anesthetic efficacy of Povicaine®. One patient even reported preference for Povicaine® over the standard anesthetic and antiseptic she previously received. Further, 18 of the 27 subjects preferred Povicaine® as a method of numbing prior to the injection. The reasons that influenced their choice of procedure as described by the patients included more comfort during the numbing process, less discomfort during the actual injection, less discomfort following the injection, and less negative impact on vision.

Povicaine® Patient Survey (n=27)
1) Have you received more than 20 eye injections?
   27—Yes
   0—No
2) Which of the following methods has been used to anesthetize (numb) your eye(s) for injections? (check all that apply)
   24—Lidocaine (numbing medicine) Gel
   20—Lidocaine-soaked Sponge
   2—An Injection of numbing medicine before the injection of medicine into your eye
   27—Povicaine® Drops
3) Comparing the methods of numbing your eye, was there any significant difference in the discomfort of the eye injection?
   26—No significant difference
   1—Yes, there was a significant difference
   If you answered “Yes”, please explain: Drops worked better
4) Which method of numbing prior to an eye injection do you prefer?
   6—No preference
   0—Gel
   3—Sponge
   0—Injection of numbing medicine
   18—Drops
If you have a preference, what are the reasons that influenced your choice:
   12—More comfortable during the numbing process (all drops)
   8/2—Less discomfort during the actual injection (2 were sponge patients; 8 were drops)
   8—Less discomfort following the injection (all drops)
   6—Less negative impact on vision (all drops)
5) If you answered “Drops” to question 4, Were you initially surprised that Drops worked as well as other methods?
   17—Yes
   1—No

### Example 4. Ophthalmic Technician Preference for Methods of Anesthetizing and Disinfecting Eyes During Ophthalmic Procedures

The impressions of experienced ophthalmic technicians in regards to the use of Povicaine® as compared to the standard anesthesia and disinfection methods were surveyed. All technicians had previously assisted or witnessed the use of both methods of anesthesia and disinfection. All technicians concurred with the reactions of patients described in Example 3 above. Technicians reported that Povicaine® is more comfortable prior to the injection, offers equivalent anesthesia during the injection, and is more comfortable following the injection. It eliminates the need to irrigate the eye, saving staff time. It eliminates the blurred vision often associated with topical anesthetic gel.

Most ophthalmic technicians reported no significant difference in perceived discomfort of the patient when Povicaine® was used, and some even reported that the patient experienced less discomfort when Povicaine® was used. The technicians explained that using lidocaine-soaked sponges (pledgets) irritated the eye and the lidocaine gel was perceived to be too thick. All technicians reported that, surprisingly, Povicaine® effectively provided adequate anesthesia for most patients receiving intravitreal injections. The survey is shown below.

Povicaine® Ophthalmic Technician Survey (n=10)
1) Have you assisted a retina specialist during at least than 1000 intravitreal injections?
   10—Yes
   0—No
2) Which of the following methods have you assisted with or witnessed being used to anesthetize eyes for intravitreal injections? (check all that apply)
   10—Lidocaine (numbing medicine) Gel
   10—Lidocaine-soaked Sponge
   10—Lidocaine subconjunctival Injection
   10—Povicaine® Drops
3) Have you assisted in at least 50 cases in which Povicaine® has been used to anesthetize an eye for an intravitreal injection?
   10—Yes
   0—No
4) Comparing the methods of anesthesia, do you perceive any significant differences in the discomfort of the eye injection?
   7—No significant difference for the majority of patients
   3—Yes, there is a significant difference
   If you answered “Yes”, please explain: (1) sponges irritate, gel too thick; (2) depends on patient; (3) sponges cause irritation, gel sticky
5) Does Povicaine® seem to provide adequate anesthesia for most patients receiving intravitreal injections?
   10—Yes
   0—No
6) Were you initially surprised that Povicaine® drops could effectively anesthetize the eye for an intravitreal injection?
   10—Yes
   0—No
7) Please indicate comments you have commonly heard from patients regarding Povicaine® vs. other methods of numbing the eye:
   4—More comfortable during the numbing process
   1—Less discomfort during the actual injection
   7—Less discomfort following the injection
   9—Less negative impact on vision
   0—None of the above

1. An ophthalmic antiseptic and anesthetic composition, the composition comprising povidone-iodine in a concentration between about 0.01% and about 5.0% by weight; and proparacaine hydrochloride in a concentration between about 0.2% and about 0.6% by weight.

2. The composition of claim 1, wherein the povidone-iodine is in a concentration of about 0.9% to about 1.1% by weight.

3. The composition of claim 1, wherein the proparacaine hydrochloride is in a concentration of about 0.4% to about 0.5% by weight.

4. The composition of claim 1, further comprising an antimicrobial preservative.

5. The composition of claim 1, further comprising a co solvent/surfactant.

6. The composition of claim 1, further comprising a viscosity increasing agent.

7. The composition of claim 1, further comprising a pharmaceutically acceptable excipient.

8. The composition of claim 1, wherein the composition is in the form of a solution, suspension, emulsion, ointment, cream, gel, or a controlled-release vehicle.

9. An ophthalmic antiseptic and anesthetic dosage form, comprising:
povidone-iodine at a concentration between about 0.01% and about 5.0% by weight;
and proparacaine hydrochloride at a concentration between about 0.2% and about 0.6% by weight.

10. The dosage form of claim 9, wherein the dosage form is eye drops.

11. The dosage form of claim 9, wherein the dosage form is a gel.

12. The dosage form of claim 9, wherein the dosage form comprises about 0.9% to about 1.1% povidone-iodine.

13. The dosage form of claim 9, wherein the dosage form comprises about 0.4% to about 0.5% of proparacaine hydrochloride.

14. (canceled)

15. A method of treatment for an eye, comprising administering a therapeutically effective amount of the ophthalmic antiseptic and anesthetic composition according to claim 1, to the eye of a subject in need thereof.

16. The method of treatment of claim 15, wherein the povidone-iodine is at a concentration of about 0.9% to about 1.1% by weight.

17. The method of treatment of claim 15, wherein the proparacaine hydrochloride is at a concentration of about 0.4% to about 0.5% by weight.

18. The method of claim 15, wherein the subject is a human subject.

19. A method of relieving discomfort associated with an ophthalmic procedure, the method comprising administering a therapeutically effective amount of the ophthalmic antiseptic and anesthetic composition according to claim 1, to the eye of a subject in need thereof.

20. A method of anesthetizing and disinfecting an eye during an ophthalmic procedure, the method comprising administering a therapeutically effective amount of the ophthalmic antiseptic and anesthetic composition according to claim 1, to the eye of a subject in need thereof.

21. A kit comprising one or more containers containing an amount of povidone-iodine in a concentration between about 0.01% and about 5.0% by weight; and an amount of proparacaine hydrochloride in a concentration between about 0.2% and about 0.6% by weight.

22-26. (canceled)
